(19) United States
(12) Patent Application Publication
(10) Pub. No.: US 2019/0125496 A1
(43) Pub. Date: May 2, 2019

(54) DENTAL ORTHOTIC AND PROTECTION DEVICES AND A KIT CONTAINING SUCH DENTAL ORTHOTIC AND PROTECTION DEVICES

(71) Applicant: Tammy Lynne Ingram, Calgary (CA)

(72) Inventor: Tammy Lynne Ingram, Calgary (CA)

(21) Appl. No.: 15/798,298

(22) Filed: Oct. 30, 2017

(51) Int. Cl. A61C 7/12; A61C 9/00; A61C 19/02; A61F 5/56; A63B 71/08

(52) U.S. Cl. CPC A61C 7/125; A61C 9/0026; A61F 2005/563; A61F 5/566; A63B 71/085; A61C 19/02

(57) ABSTRACT

Examples of a dental orthotic guard and protection device are disclosed. The device is re-usable and can be used by users with or without braces. The protection device comprises a body shaped to correspond to user's dental arch and an aerating trough formed in the body such that it rises upward from its surface. The aerating trough has a plurality of openings to allow fluid circulating in and out through the trough to prevent fluids accumulating on a surface of the protector and provide free air flow. The dental orthotic can further comprises a U-shaped guard with top and bottom surfaces that are substantially flat such that the guard sits flat on top of user's teeth to provide fluid flowing between the user teeth and the guard. The dental protector and guard can be connected as a single piece for use as dental orthotic device in small children.

### TECHNICAL FIELD

The present invention relates generally to a reusable dental orthotic and protection device and to a packing kit containing the dental orthotic and protection device.

### BACKGROUND

Unless otherwise indicated herein, the materials described in this section are not prior art to the claims in this application and are not admitted to be prior art by inclusion in this section.

Dental conditions such as bruxism (teeth grinding) and malocclusion (crooked teeth/off balance jaws) may cause many problems such as damage to teeth, damage to jaw joints, headaches, etc. It is estimated that 45 million Americans complain of headaches each year. A portion of these occurrences are experienced by adults with dental orthotics, engineered cosmetic teeth, braces, partial plates and full dentures. These statistics are much greater if one factors in the number of children and adolescents with malocclusion (crooked teeth/off balance jaws) and braces or retainers, who experience headaches and migraines. Cranial Nerve Trauma (CNT) is often caused by natural malocclusion, disease, injury and/or modern dentistry. For example, based on inventor's research Trigeminal Nerve Trauma triggered by bilateral dental imbalance can possibly cause one side of the face to appear hypertrophic while the other side appears hypotrophic, meaning one side of the face looks as if it's seizing while the other appears less responsive. In addition to the numerous facial and cranial indicators of CNT due to malocclusion there are many others such as chronic fatigue and chronic illness and pain (the trigeminal nerve alone is reported to have approximately 100× more dense pain fibers than any other nerve in the body and is a neuromodulator).

The traditional night guards are made of a hard material built on either maxillary (upper jaw) and/or mandibular (lower jaw) teeth. These are expensive devices since a dentist spends time to custom modify and adjust such devices in consideration of user's unique malocclusion. In addition, such guards feel cumbersome and do not provide proper hygiene, such as unobstructed saliva and air flow so the saliva gets stuck in the pockets or teeth trough of such devices where bacteria can build up. Some of the known dental orthotics that are used to prevent bruxism and malocclusion can also provide protection of the teeth (protective mouth piece) such as for example the one used in contact sports however such orthotics are hard to use when one uses braces since such orthotics do not fit good around the braces and teeth.

### SUMMARY

In one aspect, a dental orthotic protection device for protecting user's teeth and braces is provided. The device comprises a semicircular body shaped to correspond to user's dental arch and an aerating trough formed in the semicircular body so that it extends around a longitudinal axis from one posterior end to the other posterior end of the body. A number of openings are formed in the body allowing access to the aerated trough to allow fluid flowing through the trough. The aerated trough rises of a surface of the body and is sized to fit user's dental features.

In another aspect, a dental orthotic guard and protection device is provided. The device comprises a U-shaped dental guard and a dental protector having a body for protecting user's teeth and an aerating trough with flow openings to allow fluid flowing through the trough. A top surface and a bottom surface of the guard are substantially flat such that it sits flat on top of user's teeth to allow fluid flowing between the user teeth and the guard.

In another aspect, the top and bottom surfaces of the guard are textured forming a number of perturbations and valleys such that fluid flows throughout such valleys.

In yet another aspect, the dental guard and the dental protector are connected into a single piece integrated device that can be used in small children as dental orthotic protecting device.

In one aspect, a kit is provided that comprises a container containing a pre-fabricated U-shaped dental guard and a pre-fabricated dental protector. The kit can further comprise a pre-fabricated single-piece dental guard and protector for use in small children.

In another aspect, the kit comprises a molding composite material and an injector for injecting the molding composite material for custom made U-shaped dental guard. The kit can further comprise a molding base for a single piece dental orthotic guard and protection device such that a customized single piece dental guard and protector can be made for use in small children.

In addition to the aspects and embodiments described above, further aspects and embodiments will become apparent by reference to the drawings and study of the following detailed description.

### BRIEF DESCRIPTION OF THE DRAWINGS

Throughout the drawings, reference numbers may be re-used to indicate correspondence between referenced elements. The drawings are provided to illustrate example embodiments described herein and are not intended to limit the scope of the disclosure. Sizes and relative positions of elements in the drawings are not necessarily drawn to scale. For example, the shapes of various elements and angles are not drawn to scale, and some of these elements are arbitrarily enlarged and positioned to improve drawing legibility.

FIG. 1 is a prospective view of an example of a dental orthotic package kit containing pre-fabricated dental guard and protection devices.

FIG. 2 shows two views of a process for making a custom-made dental orthotic guard.

FIG. 3 is a front view of a custom-made dental guard and a pre-fabricated dental protector.

FIG. 4 is a perspective view of a lingual surface of the dental protector showing an aerating trough and flow openings.

FIG. 5 is a perspective view of a pre-fabricated dental protector showing a number of openings that allow fluid flow through the dental protector.

FIG. 6 shows two views of an integrated single piece dental orthotic guard/protector intended for use in small children.

FIG. 7 is a perspective view of an example of a dental orthotic package kit including pre-fabricated dental guard and protector, injection composite for molding custom-made dental guard and protector and a junior orthotic guard/protector.

### DETAILED DESCRIPTION OF SPECIFIC EMBODIMENTS

The present invention discloses a thin, malleable dental grade removable orthotics to be used as teeth/braces protection piece and grinding protection piece. The dental orthotics of the present invention are cost-effective reusable dental appliances for retail and professional use that can be used by individuals that suffer from headaches, sleep apnea, chronic pain, during a childbirth labor or for any other inconsistent pain management, or as an aid for establishing neuromusculoskelatal equilibrium. For example, with time teeth may slightly shift and erode in an effort to stabilize body equilibrium and the dental orthotic devices of the present invention can help in a process to prevent teeth erosion and may prevent any pain and discomfort associated with such shift. The dental orthotics of the present invention can be pre-fabricated or can be custom-made using pieces of a dental orthotic kit of the present invention.

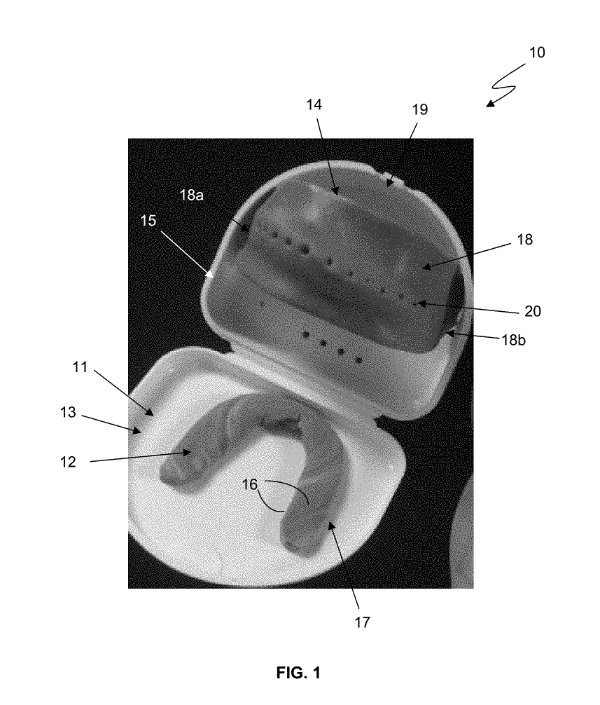

FIG. 1 illustrates a dental orthotic kit 10 comprising an example of a dental orthotic guard 12 and a dental orthotic protector 14. The dental guard 12 and protector 14 can be made of a medical grade material that is strong and malleable, such as for example, any medical grade plastic, silicone or rubber. The selected material can also prevent absorption of mouth fluids or cleaning fluids. The guard 12 has a U-shaped body that corresponds to user's dental arch and has a top and a bottom surface 16, and a rounded edge 17. The bottom surface 16 can sit over the teeth of the lower jaw while the teeth of the upper jaw can rest on the top surface 16 of the guard 12 when the mouth of the user is closed. In one implementation, the top and the bottom surfaces 16 are substantially flat such that the guard 12 sits flat between user's teeth to allow a fluid (saliva and air) to easy flow in the space between the user's teeth and the guard. This can potentially prevent bacteria building up in pools or pockets formed in some known prior art dental guard that include dental trench for accommodating teeth of the upper/lower jaws. The upper and lower jaws can freely slide from side to side or forward and back as needed, so if one is grinding it support the movement eliminating the negative effect of grinding (e.g. during teeth likely slight shift to stabilize body equilibrium). In addition, if the user has a bad bite (malocclusion) that is locked into place by crooked or engineered teeth, it may help to eliminate the anatomy by replacing it with a smooth surface which allows more natural range of motion. In another implementation, the one or both of the surfaces 16 can be textured, for example can have a number of perturbations (e.g. ribs or bumps) rising of the top and the bottom surfaces 16, thus forming a textured surface with a number of crests and valleys, so that a fluid can flow throughout the formed valleys.

The dental protector 14 can have a semicircular body 18 that is shaped to correspond to user's dental arch. The protector 12 can be sized to extend over the teeth and pass gum line and can be secured and held over the teeth by user's upper and lower lips. The body 18 can have a facial surface and a lingual surface that faces user's teeth and gum. A plurality of openings 20 that are formed in the body 18 can extend between its facial and lingual surfaces thus allowing any fluid (i.e. saliva or air) to flow through the openings 20. This can provide better hygiene since will prevent fluid, such as saliva, from accumulating in any pocket formed on the facial or lingual surface of the protector 14 and can allow free mouth breathing for people that have problem breathing through the nose (e.g. people that suffer from allergy and/or sinuses). In one embodiment, the protector 14 can comprise a mesh extending from one posterior end 18a to the other posterior end 18b forming the openings 20. The facial and lingual surface of the dental protector 14 needs to be smooth and non-irritating to the soft tissues of the mouth.

The kit 10 can further comprise a container 11 with a bottom 13, a lid 15 and a fastener 19 to close and open the container 11. The container can be a box shaped or can have any other suitable shape and size and can be made of any suitable strong material. The fastener can be a snapping fastener (as the one illustrated in FIG. 1) with a tab formed on the lid 15 and a corresponding notch formed on the bottom 13 or any other suitable fastener without departing from the scope of the invention.

The dental guard 12 and the dental protector 14 can be pre-fabricated and the user or dental practitioner can only trim the excess material to a desired size. In one implementation, the kit 10 can include a trimmer for trimming the guard 12 and/or the protector 14 to a desired size. This can result in significant cost savings over similar devices that require use of mold models and the services of a dental laboratory in their construction.

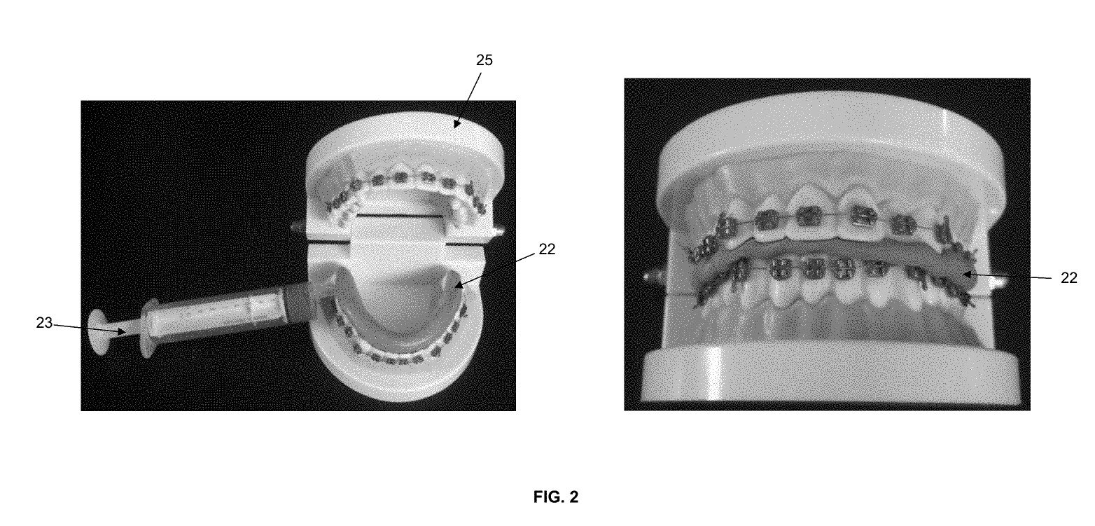

In one implementation, the guard 12 and/or the protector 14 can be custom made. FIG. 2 shows a custom made dental guard 22. An injector 23 can be used for injecting a molding composite for custom made U-shaped dental guard 22. For example, the composite material can be dental grade material, such as vinyl polysiloxane impression material. The molding material can be injected in the user's mouth using the injector 23. For example, the molding material can be put over the teeth of the lower jaw (photo on the left) and when the user closes his/her mouth (photo on the right) the guard can be easily molded to conform to the configuration of the user's upper and lower teeth. Thus, the guard 22 can be easily molded in the user's mouth to conform to the configuration of the user's teeth, saving time and costs, since it does not require services of a dental laboratory.

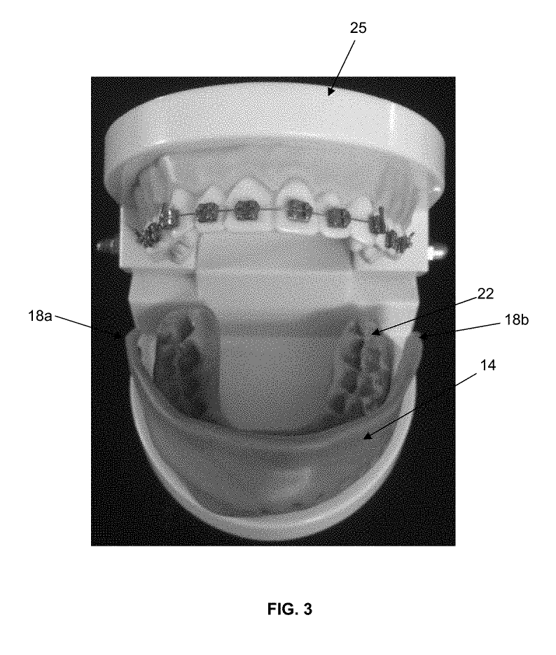

In some embodiment, a dental model 25 of user's jaw and teeth can be used for making custom dental guard 22. FIG. 3 shows an example of a custom made dental guard 22 and a pre-fabricated protector 14. Person skilled in the art would understand that the protector 14 can also be custom-made without departing from the scope of the invention. Using a custom made dental orthotic guard 22, the user can set the bite with their jaw more down, forward, left and/or right for a better balanced position. Some users can find relief by locking their jaw into a new (balanced) custom dentation bite provided by the custom made guard 22 while others may feel better by eliminating the bite and grinding with the prefabricated thin or ultra-thin flat dental orthotic guard 12.

In one implementation the dental guard 12, 22 can comprise means for securing the guard in place when in use. For example, a portion of the edge 17 of the dental guard 12, 22 can be slightly contoured (raised in direction toward the teeth of upper or lower jaws), so that it can tightly grip at least one tooth on each posterior side of the user's lower or upper jaw (depending whether the guard 12, 22 is secured to the teeth of the lower or upper jaw). In one implementation, the entire edge 17 can be contoured, so that it completely grips the teeth of either lower or upper jaw. In such case, the countered part of the edge can comprise a plurality of slits (not shown) to allow fluid to flow easily through the slits. Any other suitable means for securing the guard in place can be used without departing from the scope of the invention.

The wall of the protector's body 18 can be thin so it can be comfortable to use for a certain time period. In some embodiments the protector 14 can be made of a stronger, resilient, material and/or the wall of the protector's body can be thicker to provide the necessary protection required for, for example, contact sports. The guard 12, 22 can also be a thinner or thicker, and both the guard 12, 22 and the protector 14 can be trimmed to size.

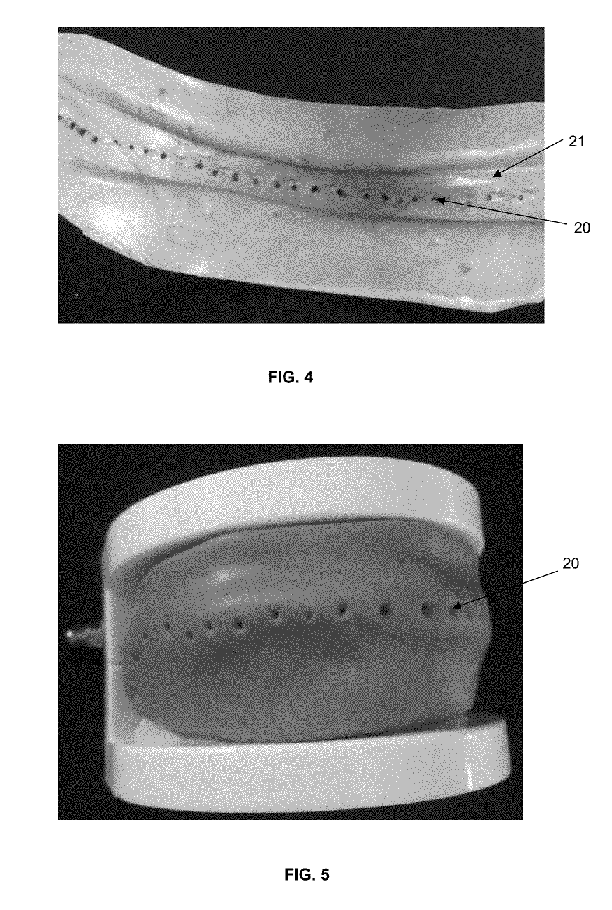

FIGS. 4 and 5 show an example of a pre-fabricated dental protector 14. The protector 14 can be designed to be used by persons that wear braces or users without braces. An aerating trough 21 can be formed in the body 18 that rises up of the surface of the body 18, such that it forms a depression in the lingual surface. It can extend around longitudinal axis of the body 18 (middle line of the protector 14) from one posterior end 18a to the other posterior end 18b, such that it can envelop user's braces on the upper and lower jaws teeth (allow enough space for the braces) and thus provides a protection for the teeth and the braces as well as easy flow of air and saliva. The aerating trough 21 can be sized to fit any other dental features that user may have, such as for example, provide enough space for any crooked tooth. In addition, the protector 14 can be used by users (with or without braces/crooked teeth) that prefer breathing through mouth (“mouth breathers”). The plurality of flow openings 20 that are in fluid communication with the aerating trough 21 can allow the air (and saliva) to flow easily through the trough 21. So, mouth breathers (e.g. persons suffering from sinus/allergy or any other mouth breathers) can freely breathe through the openings 20 and aerating trough 21 without need to remove the protector 14.

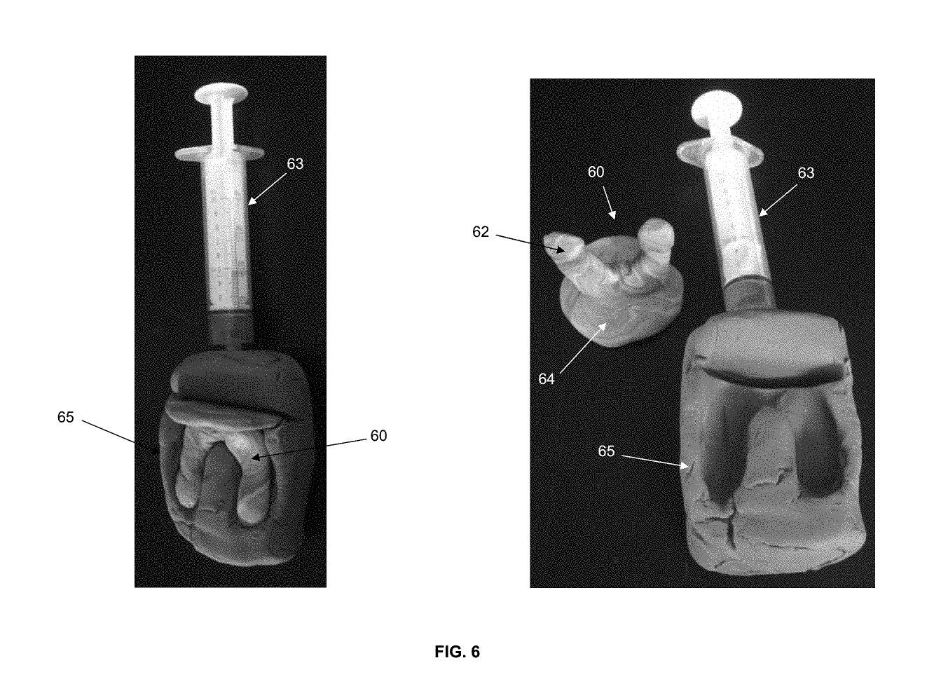

In the examples of dental guard and protecting devices illustrated and described with respect to FIGS. 1-5, the guard 12, 22 and the protector 14 are two separate pieces that can be used independently or in combination. For example, during night one can use only the guard 12, 22 or can use both the guard 12, 22 and the protector 14, so that the jaw is held in a more natural position which can bring relief and can result in better sleep and less headaches and pains. Both dental guard 12 and the protector 14 are re-usable meaning they can be used multiple times with proper hygiene (cleaning). Having something in the mouth helps generate a natural reaction to breathe through the nose with the mouth closed which may help for sleep apnea. In one implementation, the dental guard and the dental protector can be connected and integrated into a single piece dental guard and protector. FIG. 6 shows a single piece integrated dental guard and protector 60 designed to be used in small children. The integrated dental guard and protector 60 can comprise a U-shaped guard 62 that is sized and shaped to conform to a dental arch/gum line contour. The guard 62 is connected to a dental protector 64. The protector 64 can be similar to the protector 14, and can extend over the teeth or gum or it can extend over the upper and lower lips as a shield. A top and a bottom surface of the guard 62 can be flat or textured (as described herein above). A plurality of flow openings (not shown) can be provided in the body of the protector 64 to allow fluid (e.g. air) flow. The integrated dental guard and protector 60 can be used in small children (toddlers) as replacement to, for example a baby soother, a pacifier or teething rings. The dental guard and protector 60 distributes bite force evenly, increases blood circulation (which may help with any pain/discomfort), encourages chewing instead of sucking and works as a shield/guard against falls and accidents. It can be made of a strong, slick and malleable composite rubber material. In some implementations, a prefabricated mold base 65 can be designed for making a custom made integrated dental guard and protector 60. A molding composite material can be injected by an injector 63 into the molding base 65 to make the custom made single piece dental guard and protector 60. The pre-fabricated single integrated dental guard and protector 60 and/or the molding base 65 for the custom-made one can come in various sizes so that children of different age (infants to preschoolers i.e. 0-6 years old) and different jaw size can use it.

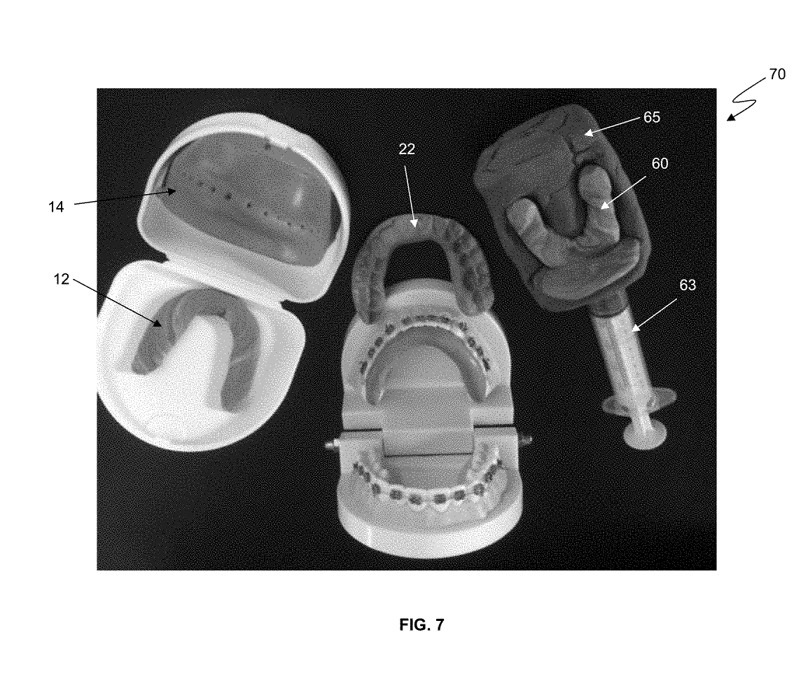

FIG. 7 illustrated another example of a package kit 70 that can comprise pre-fabricated guard 12 and pre-fabricated protector 14 as one described with respect to FIG. 1, as well as a composite material and molding injector 23, 63 for making a custom made guard 22 and/or protector (as described with respect to FIGS. 2 and 3). The kit 70 can further comprise a single piece integrated dental guard and protector 60 that can be pre-fabricated or custom-made. In case of a custom-made dental guard and protector 60 a molding base (model) 65 can also be provided for making such custom made single piece dental guard and protector. The molding base 65 for the custom-made single piece dental guard and protector can come in specified size for making custom-made and custom-sized single piece dental guard and protector. In one implementation, the kit 10 and 70 can comprise an instruction booklet to instruct the user how to use the guard and the protector or how to trim it to adjust the size or how to make a custom made dental guard and dental protector.

While particular elements, embodiments and applications of the present disclosure have been shown and described, it will be understood, that the scope of the disclosure is not limited thereto, since modifications can be made without departing from the scope of the present disclosure, particularly in light of the foregoing teachings. Thus, for example, in any method or process disclosed herein, the acts or operations making up the method/process may be performed in any suitable sequence and are not necessarily limited to any particular disclosed sequence. Elements and components can be configured or arranged differently, combined, and/or eliminated in various embodiments. The various features and processes described above may be used independently of one another, or may be combined in various ways. All possible combinations and sub-combinations are intended to fall within the scope of this disclosure. Reference throughout this disclosure to “some embodiments,” “an embodiment,” or the like, means that a particular feature, structure, step, process, or characteristic described in connection with the embodiment is included in at least one embodiment. Thus, appearances of the phrases “in some embodiments,” “in an embodiment,” or the like, throughout this disclosure are not necessarily all referring to the same embodiment and may refer to one or more of the same or different embodiments.

Various aspects and advantages of the embodiments have been described where appropriate. It is to be understood that not necessarily all such aspects or advantages may be achieved in accordance with any particular embodiment. Thus, for example, it should be recognized that the various embodiments may be carried out in a manner that achieves or optimizes one advantage or group of advantages as taught herein without necessarily achieving other aspects or advantages as may be taught or suggested herein.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without operator input or prompting, whether these features, elements and/or steps are included or are to be performed in any particular embodiment. No single feature or group of features is required for or indispensable to any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

Conjunctive language such as the phrase “at least one of X, Y and Z,” unless specifically stated otherwise, is otherwise understood with the context as used in general to convey that an item, term, etc. may be either X, Y or Z. Thus, such conjunctive language is not generally intended to imply that certain embodiments require at least one of X, at least one of Y and at least one of Z to each be present.

The example calculations, simulations, results, graphs, values, and parameters of the embodiments described herein are intended to illustrate and not to limit the disclosed embodiments. Other embodiments can be configured and/or operated differently than the illustrative examples described herein.

What is claimed is:

1. A dental orthotic protection device for protecting user's teeth, the device comprising:
a semicircular body shaped to correspond to user's dental arch and sized to extend over the teeth and pass gum line;
an aerating trough formed in the semicircular body raising of a surface of the body and extending around the longitudinal axis from one posterior end to the other posterior end of the body; and
a plurality of openings formed along the aerating trough to allow fluid flowing through the openings and trough.

2. The dental protection device of claim 1, wherein the aerating trough is sized to fit user's braces or any dental feature.

3. The dental protection device of claim 1 being custom-made to fit user's unique dental features.

4. A dental orthotic guard and protection device, the device comprising:
a U-shaped dental guard having a top surface and a bottom surface and a rounded edge, the dental guard sized and shaped to correspond to user's dental arch; and
a dental protector having a body with an aerating trough formed in the body and extending around a longitudinal axis from one posterior end to the other posterior end of the body, the aerating trough raising of a surface of the body and having a plurality of openings to allow a fluid flowing through the openings and the trough.

5. The device of claim 4, wherein the dental protector is separate and independent from the dental guard.

6. The device of claim 4, wherein the body is semi-circularly shaped to correspond to user's dental arch and sized to extend over the teeth and pass gum line, the aerating trough being sized to fit user's braces or any dental feature.

7. The device of claim 4, wherein the top and bottom surfaces of the guard are substantially flat such that it sits flat on top of user's teeth to provide fluid flowing between the user's teeth and the guard.

8. The device of claim 4, further comprising a number of perturbations rising of the top and the bottom surfaces forming a textured surface with a number of crests and valleys, wherein a fluid flows through out such valleys.

9. The device of claim 7, wherein the dental guard and the protector are connected forming a single piece integrated device, the single piece integrated dental guard and protector being used in small children.

10. The device of claim 8, wherein the dental guard and the protector are connected forming a single piece integrated device, the single piece integrated dental guard and protector being used in small children.

11. The device of claim 9, wherein the single piece dental guard and protector having a pre-determined size to fit different age group children.

12. The device of claim 10, wherein the single piece dental guard and protector having a pre-determined size to fit different age group children.

13. The device of claim 4, wherein the edge of the dental guard is contoured such that it provides a grip to at least one tooth on each posterior side of the user.

14. A kit for a dental orthotic guard and protection device, the kit comprising:
a container having a bottom, a lid and a fastener to close the container;
a U-shaped dental guard having a top surface and a bottom surface and a rounded edge, the dental guard sized and shaped to correspond to user's dental arch; and
a dental protector having a semicircular body with an aerating trough formed in the body, raising of a surface of the body and having a plurality of openings to allow fluid flowing through the openings and the trough, the semicircular body being shaped to correspond to user's dental arch and sized to extend over the teeth and pass gum line, the dental protector being separated and independent from the dental guard.

15. The kit of claim 14, further comprising an additional dental guard connected to a protector forming a single piece integrated dental guard and protector, the integrated dental guard and protector being used in small children.

16. The kit of claim 14, further comprising a molding composite material and an injector for injecting the molding composite for a custom made dental orthotic guard and protector.

17. The kit of claim 16, further comprising a molding base for a single piece dental orthotic guard and protection device for a custom made single piece dental orthotic guard and protector.

18. The kit of claim 14, further comprising a trimming device to trim the dental guard and protector to a desired size.

19. The kit of claim 14, further comprising instruction booklet.
